(19) United States
(12) Patent Application Publication
(10) Pub. No.: US 2009/0105490 A1
(43) Pub. Date: Apr. 23, 2009

(54) Polymorphic forms of ramelteon and processes for preparation thereof

(76) Inventors: Nurit Perlman, Kfar Saba (IL); Michal Rafilovich, Petach-Tikva (IL); Rinat Moshkovits-Kaptsan, Raanana (IL); Elena Ben Moha-Lerman, Kiryat Ono (IL); Revital Lifshitz-Liron, Hertzlia (IL)

Correspondence Address: KENYON & KENYON LLP, ONE BROADWAY, NEW YORK, NY, 10004, US

(21) Appl. No.: 12/218,369

(22) Filed: Jul. 14, 2008

Related U.S. Application Data
(60) Provisional application No. 60/959,405, filed on Jul. 12, 2007.
(60) Provisional application No. 61/073,221, filed on Jun. 17, 2008.

(51) Int. Cl. C07D 307/77

(52) U.S. Cl. 549/458

(57) ABSTRACT

An amorphous form of ramelteon is provided, as well as mixtures of amorphous and crystalline ramelteon. Also provided are methods of preparing amorphous ramelteon and mixtures of amorphous and crystalline ramelteon, pharmaceutical compositions comprising amorphous ramelteon and mixtures of amorphous and crystalline ramelteon, and methods of treatment of insomnia using the compositions of the invention.

### FIELD OF THE INVENTION

The present invention relates to the solid state chemistry of ramelteon.

### BACKGROUND OF THE INVENTION

(S)-N-[2-(1,6,7,8-tetrahydro-2H-indeno-[5,4-b]furan-8-yl)ethyl]propionamide, whose international nonproprietary name is ramelteon, has the following chemical structure.

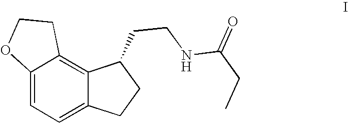

Ramelteon, CAS No. 196597-26-9, is a melatonin receptor agonist with both high affinity for melatonin MT1 and MT2 receptors and selectivity over the MT3 receptor. The empirical formula for ramelteon is C₁₆H₂₁NO₂, and its molecular weight is 259.34. Ramelteon is freely soluble in methanol, ethanol, DMSO, and 1-octanol, and slightly soluble in water and aqueous buffers.

Ramelteon is the active ingredient in trade marked ROZEREM™, and is approved by the United States Food and Drug Administration for the treatment of insomnia characterized by difficulty with sleep onset. Different processes for preparing ramelteon are disclosed in U.S. Pat. No. 6,034,239, JP 11080106, JP 11140073 and WO 2006/030739. The X-ray powder diffraction pattern of a crystalline form of ramelteon is disclosed in IP.com publication with code number IPCOM000160609D.

The present invention relates to the solid state physical properties of ramelteon. These properties can be influenced by controlling the conditions under which ramelteon is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular polymorphic form of a substance. The polymorphic form may give rise to thermal behavior different from that of the amorphous material or another polymorphic form.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Thus, there is a need in the art for the discovery of additional polymorphic forms of ramelteon.

### SUMMARY OF THE INVENTION

The present invention encompasses amorphous ramelteon, and mixtures of amorphous ramelteon and crystalline Form A of ramelteon.

One embodiment of the present invention encompasses a mixture of amorphous and Form A of ramelteon, having a PXRD pattern substantially as depicted in FIG. 1.

The present invention further encompasses processes for preparing amorphous ramelteon and mixtures of amorphous and Form A ramelteon.

The present invention encompasses a substantially amorphous form of ramelteon.

The present invention further encompasses processes for preparing a substantially amorphous form of ramelteon.

The present invention further encompasses novel processes for preparing Form A of ramelteon.

The present invention further encompasses a pharmaceutical composition comprising a mixture of amorphous and Form A of ramelteon and at least one pharmaceutically acceptable excipient.

The present invention further encompasses a process for preparing a pharmaceutical formulation comprising a mixture of amorphous and Form A of ramelteon with at least one pharmaceutically acceptable excipient.

The present invention further encompasses the use of a mixture of amorphous and Form A of ramelteon for the manufacture of a pharmaceutical composition.

The present invention further encompasses a pharmaceutical composition comprising a substantially amorphous form of ramelteon form and at least one pharmaceutically acceptable excipient.

The present invention further encompasses a process for preparing a pharmaceutical formulation comprising a substantially amorphous form of ramelteon form with at least one pharmaceutically acceptable excipient.

The present invention further encompasses the use of a substantially amorphous form of ramelteon form for the manufacture of a pharmaceutical composition.

### BRIEF DESCRIPTION OF THE FIGURES

FIG. 1 shows a powder X-ray diffraction pattern for a mixture of amorphous and Form A of ramelteon

FIG. 2 shows a powder X-ray diffraction pattern for a substantially amorphous form of ramelteon.

FIG. 3 shows a powder X-ray diffraction pattern for ramelteon Form A.

### DETAILED DESCRIPTION

As used herein, “ramelteon” used as starting material may be prepared according to any of the methods described in the literature, including U.S. Pat. No. 6,034,239, JP 11080106,

As used herein, room temperature refers to a temperature of about 20° C. to about 30° C.

As used herein, relative volume (“V”) refers to ml per gram. For example, 30V means 30 ml solvent per one gram of compound.

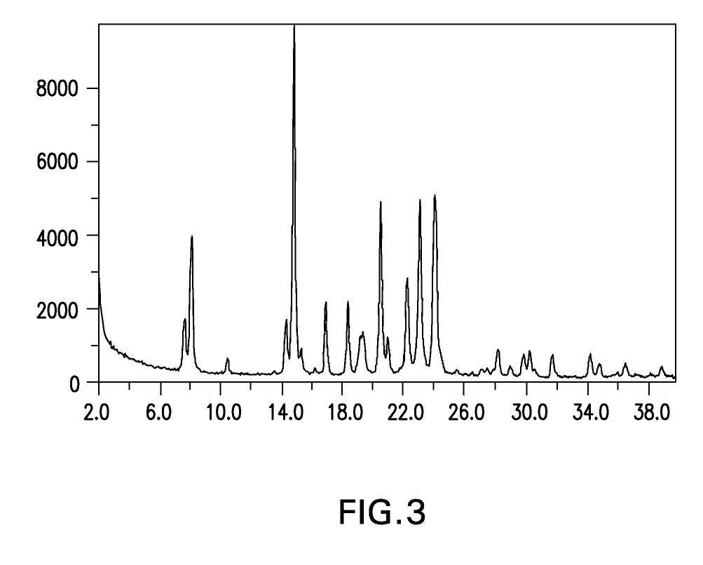

The present invention presents ramelteon Form A. Form A is characterized by a powder XRD pattern with peaks at about 7.6, 8.0, 14.2, 14.7 and 16.8±0.2 degrees two-theta. Preferably, this form is further characterized by powder XRD pattern with one or more peaks selected from the list consisting of about 18.3, 22.2 and 23.0±0.2 degrees two-theta, or substantially as depicted by a powder XRD pattern substantially as depicted in FIG. 3.

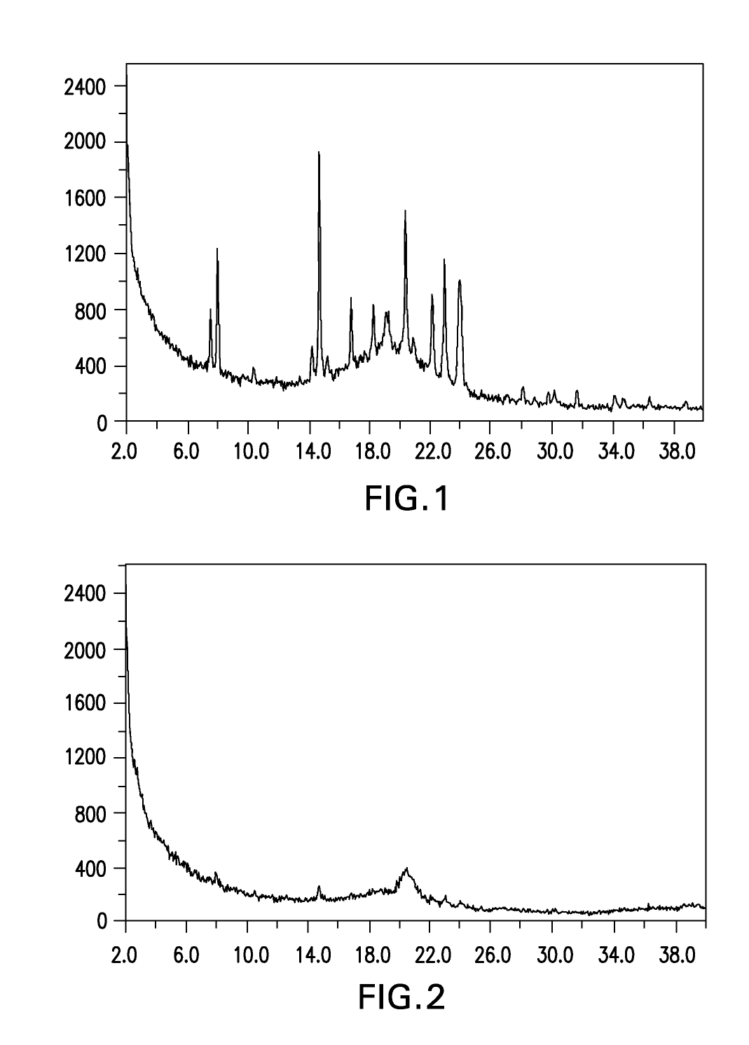

One embodiment of the present invention encompasses a mixture of amorphous and Form A of ramelteon, having a PXRD pattern substantially as depicted in FIG. 1.

The amount of crystallinity is quantified by a “crystallinity index” which can be calculated by most Powder X-Ray Diffraction software. Preferably, according to this quantification, the mixture of amorphous and Form A of ramelteon is in a ratio of 20%-80% to 60%-40% Form A to amorphous form.

The present invention encompasses processes for preparing a mixture of amorphous and Form A of ramelteon comprising slurrying ramelteon in n-decane.

Preferably, the mixture is maintained at about 20° C. to about 30° C. for about 1 hour to about 24 hours. The wet material can be recovered using any method known in the art, for example it can be isolated by decantation or filtration.

In another embodiment, the present invention presents a substantially amorphous form of ramelteon form.

As used herein, “substantially amorphous” form refers to an amorphous form which contains less than about 40% crystalline Form A. Preferably, the substantially amorphous form has less than about 30% and even more preferably less than about 20% crystalline form A. More preferably still, the substantially amorphous form contains less than about 10%, or less than about 5%, and more than 1% by weight of crystalline Form A.

In one embodiment, the present invention presents a substantially amorphous form of ramelteon form, having a PXRD pattern substantially as depicted in FIG. 2.

In another embodiment, the present invention encompasses a substantially amorphous form of ramelteon, characterized by a PXRD pattern with peaks at about 8.0, 14.7, 20.4 and 23.0±0.2 degrees two-theta.

In another embodiment, the present invention encompasses processes for preparing the above-mentioned substantially amorphous form of ramelteon comprising slurrying ramelteon in a mixture of water and propylene glycol monomethyl ether (PGME).

Preferably, the ratio of PGME to water is 1:1 up to 1:10. Preferably, the mixture is maintained at about 70° C. to about 100° C., preferably at about 75° C. to about 85° C., for about 1 hours to about 10 hours and optionally another 16-48 hours at about 20-30° C. The wet material can be recovered using any method known in the art, for example it can be isolated by decantation or filtration.

The present invention further presents novel processes for preparing Form A of ramelteon.

In one embodiment, the invention encompasses a process for preparing Form A of ramelteon comprising crystallizing of ramelteon from an organic solvent selected from the group consisting of propylene glycol monomethyl ether, propylene glycol, dimethyl formamide (DMF), acetic acid, isopropanol (IPA), acetone, methanol, ethanol, dimethyl sulfoxide (DMSO), tetrahydrofuran and acetonitrile (MeCN), followed by the addition of water to induce precipitation of the obtained Form A.

Preferably, ramelteon is introduced to the organic solvent at a temperature of about 20° C. to about 30° C. and the mixture is maintained for a sufficient period of time to give clear solution. A clear solution may be obtained after about 1 minute to about 40 minutes, or about 16 to about 65 hours. Preferably, the relative volume of the water 0.5V-3V. The obtained Form A can be recovered from the mixture by any method known in the art. Preferably, it is isolated by vacuum filtration.

In another embodiment, the present invention provides yet another process for preparing Form A of ramelteon comprising crystallizing of ramelteon from an organic solvent. The organic solvent is selected from the group consisting of ethyl acetate, acetonitrile, dimethyl carbonate, and diethyl carbonate.

Preferably, the ramelteon is dissolved in the organic solvent at a temperature of about 20° C. to about 100° C., preferably about 20° C. to about 85° C., more preferably about 20° C. to about 80° C.

Preferably, the mixture of ramelteon and an organic solvent is maintained at a temperature of about −10° C. to about 40° C., preferably about −5° C. to about 30° C., more preferably about 0° C. to about 25° C. for about 5 min to about 24 hours, preferably about 10 min to about 1 hour, for example, about 15 min, about 30 min, about 40 min or about 1 hour. The obtained product is recovered from the mixture using any method known in the art, most preferably, it is filtered. Optionally, the ramelteon Form A is dried at oven at a temperature of about 30° C. to about 60° C., for example, about 40° C. to about 60° C., more preferably about 35° C. to about 55° C., yet more preferably about 40° C. to about 50° C. for about 10 hours to about 30 hours, more preferably about 16 hours to about 25 hours, more preferably about 16 hours to about 22 hours.

In another embodiment, the present invention provides yet another process for preparing Form A of ramelteon comprising crystallizing ramelteon from a mixture of an organic solvent and an anti solvent. The organic solvent is selected from the group consisting of ethyl acetate, ethanol, toluene, acetone, isopropanol, methyl isobutyl ketone (MIBK), dichloromethane (DCM), chloroform, tetrahydrofuran, dimethyl sulfoxide (DMSO), and mixture thereof. The anti solvent can be selected form a group consisting of n-heptane, diethyl ether, diisopropyl ether, methyl tert-butyl ether, and methylcyclohexane (MeCycHx).

As used herein, an anti-solvent is a liquid that when added to a solution of ramelteon in a solvent, induces precipitation of ramelteon. Precipitation of ramelteon is induced by the anti-solvent when addition of the anti-solvent causes ramelteon to precipitate from the solution, or to precipitate more rapidly, or to precipitate to a greater extent than ramelteon would precipitate out of the solvent without the anti solvent.

In one specific embodiment, the mixture of solvents and anti solvents can be selected from the group consisting of: toluene/n-heptane, acetone/n-heptane, isopropanol/n-heptane, isopropanol/diethyl ether, ethyl acetate/diethyl ether, methyl isobutyl ketone/n-heptane, acetone/diisopropyl ether, methyl isobutyl ketone/diethyl ether, dichloromethane/methyl cyclohexane, chloroform/methyl cyclohexane, tetrahydrofuran/methyl tert butyl ether, ethanol and ethyl acetate/methyl cyclohexane, and toluene and dimethyl sulfoxide/methyl cyclohexane.

Preferably, the ramelteon is dissolved in the organic solvent at a temperature of about 20° C. to about 100° C., preferably about 20° C. to about 85° C., more preferably about 20° C. to about 80° C.

Preferably, the mixture of ramelteon, an organic solvent, and an anti solvent is maintained at a temperature of about −10° C. to about 40° C., preferably about −5° C. to about 30° C., more preferably about 0° C. to about 25° C. for about 5 min to about 24 hours, preferably about 10 min to about 17 hours, for example, about 15 min, about 30 min, about 40 min or about 1 hour. The obtained product is recovered form the mixture using any methods known in the art, most preferably, it is filtered. Optionally, the ramelteon Form A is dried at oven at a temperature of about 30° C. to about 60° C., for example, about 40° C. to about 60° C., more preferably about 35° C. to about 55° C., yet more preferably about 40° C. to about 50° C. for about 10 hours to about 30 hours, more preferably about 16 hours to about 25 hours, more preferably about 16 hours to about 22 hours.

In another embodiment, the present invention encompasses another process for preparing Form A of ramelteon comprising providing a solution of ramelteon in at least one organic solvent, and removing the solvent by spray drying.

The term “spray drying” broadly refers to processes involving breaking up liquid mixtures into small droplets (atomization) and rapidly removing solvent from the mixture. In a typical spray drying apparatus, there is a strong driving force for evaporation of solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, a typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The ramelteon product produced by spray-drying may be recovered by techniques commonly used in the art, such as using a cyclone or a filter.

The ramelteon in the solution may be any crystalline or other form of ramelteon, including various solvates and hydrates, as long as Form A of ramelteon is produced during the spray drying process of the invention. When in solution, the crystalline form of the starting material does not affect the final result since the original form is lost.

Suitable organic solvents include at least one of dichloromethane, chloroform, tetrachloromethane, a C₁-C₈ alcohol, such as ethanol, isopropanol, 2,2-dimethylethanol, or methanol, a C₃-C₈ ketone such as acetone or methyl isobutyl ketone (MIBK), a C₄-C₈ ether such as tetrahydrofuran, methyl tert butyl ether (MTBE) or dioxane, a C₃-C₈ ester such as ethyl acetate, butyl lactate or ethyl lactate, a C₁-C₃ aliphatic nitrile such as acetonitrile, and C₂-C₆ aliphatic amines such as dimethylamine. Preferred alcohols include methanol, ethanol, and propanol. Preferred ketones include acetone and methyl ethyl ketone. Preferred nitriles include acetonitrile. The more preferred solvents are ethanol, isopropanol, acetonitrile, methanol, acetone, methyl tert butyl ether (MTBE), dichloromethane, chloroform, tetrachloromethane, tetrahydrofuran, and ethyl acetate; most preferably, methanol or ethanol. The amount of the solvent used is between about 3V and about 50V relative to ramelteon.

Ramelteon can be present in any amount that will produce Form A of ramelteon upon spray drying. Preferably, the ramelteon is present in an amount of about 1% to about 30% by weight of the organic solvent, more preferably about 1% to about 20%, more preferably about 1% to about 10%, and most preferably about 2% to about 7% by weight of the solvent. One skilled in the art would understand that depending on the choice of solvent, the amount of ramelteon used may be varied. For example, when the solvent is methanol, a preferred range for the weight of ramelteon is from about 2% to about 7% of the weight of methanol, i.e. from about 2 to about 7 gram ramelteon per 100 gram methanol.

The solution may be heated to dissolve the ramelteon. The temperature suitable for dissolving ramelteon depends on the organic solvent used and the amount of ramelteon in the solution. Typically, the solution is heated to a temperature from at least about 30° C. to about reflux. Preferably, the solution is heated to about 40° C. to about 65° C., and more preferably to about 40° C. to about 50° C. The solution may be prepared at other suitable temperatures as long as the ramelteon is sufficiently dissolved. Increasing the amount of ramelteon would generally require the use of higher temperatures. Routine experimentation will provide the approximate range of suitable temperatures for a given organic solvent and amount of ramelteon. Typically the outlet temperature is about 20° C. to about 30° C., and the inlet temperature is about 40° C.

After the ramelteon is dissolved, the solution may optionally be cooled to about room temperature, or about 25° C.

In another embodiment, the present invention encompasses yet another process for preparing Form A of ramelteon comprising providing a mixture of ramelteon and an organic solvent, and removing the solvent by lyophilization.

Typically, lyophilization is done by a process comprising cooling the solution to obtain a cooled mixture, and evaporating the solvent while maintaining the mixture cooled at low temperature.

Preferably, the solution is cooled to a temperature of about 25° C., and then to about −30° C., providing the cooled mixture, which is frozen mass.

Typically, the frozen mass is then subjected to a pressure of less than about one atmosphere, to remove the solvent.

Suitable solvents include, but are not limited to, tert-butanol, a mixture of tert-butanol and water, and acetic acid and its aqueous solutions.

In another embodiment, the present invention encompasses another process for preparing Form A of ramelteon comprising dissolving ramelteon in a first organic solvent such as methyl acetate, ethyl acetate, methanol, isopropanol, acetone, methyl isobutyl ketone, or dichloromethane, followed by the evaporation of the solvent to obtain Form A of ramelteon. Alternatively, a second solvent such as diisopropyl ether, n-heptane, diethyl ether, methyl tert-butyl ether, and water may be introduced to the mixture before the evaporation.

Preferably, the mixture is maintained at a temperature of about 20° C. to about 30° C., preferably about 25° C., for about 5 min to about 72 hours, for example, about 5 min to about 48 hours, more preferably about 10 min to about 50 hours, yet more preferably about 15 min to about 48 hours, before evaporating the solvent. Optionally, the obtained Form A of ramelteon is dried at a temperature of about 30° C. to about 60° C., for example, about 40° C. to about 60° C., more preferably about 35° C. to about 50° C., yet more preferably about 40° C. to about 50° C. for about 10 hours to about 30 hours, preferably about 15 hours to about 50 hours.

In another embodiment, the present invention encompasses another process for preparing Form A of ramelteon comprising grinding ramelteon in the presence of a solvent selected form the group consisting of water, ethanol, isopropanol, and n-heptane.

The term “grinding” broadly refers to crushing a compound, typically using a mortar and pestle.

In another embodiment, the present invention encompasses another process for preparing Form A of ramelteon comprising granulating ramelteon in the presence of a solvent selected from the group consisting of water, ethanol, isopropanol, and n-heptane.

The term “granulation” broadly refers to a process comprising mixing the solid with a minimal amount of solvent, and stirring the mixture at about room temperature for the time needed to cause the desired transformation. A mechanical stirrer can be used in the process. Typically, about 0.1 to about 0.2 ml of solvent is used per 1 gram of compound. Preferably, the mixture is granulated using a rotary evaporator.

In another embodiment, the present invention encompasses yet another process for preparing Form A of ramelteon comprising melting ramelteon by heating, and then allowing the ramelteon to solidify upon cooling.

Preferably, the ramelteon is heated to about 113° C. to about 115° C. Optionally, the ramelteon is cooled at about 0° C. to about 25° C., for example, to about 0° C., before collecting the obtained ramelteon Form A. Preferably, the heating of the ramelteon is obtained by heating a flask containing ramelteon to about 123° C. to about 125° C.

In another embodiment, the present invention encompasses yet another process for preparing Form A of ramelteon comprising slurrying ramelteon in an organic solvent such as methyl ethyl ketone, dioxane, ethanol, 1-propanol, ethoxyethanol, methyl isobutyl ketone, isopropyl methyl ketone, N-methylpyrrolidone, or dimethyl carbonate followed by evaporation of the solvent to give ramelteon Form A.

methyl ethyl ketone, dioxane, ethanol, 1-propanol, ethoxyethanol, methyl isobutyl ketone, isopropyl methyl ketone, N-methylpyrrolidone, and dimethyl carbonate

Preferably, the mixture is maintained at a temperature of about 20° C. to about 30° C., preferably about 25° C. for about 1 to about 100 hours, preferably about 10 to about 80 hours, more preferably about 18 hours to about 65 hours, before evaporation. If dimethyl carbonate is used, the mixture is maintained at about 70° C. to about 100° C., preferably at about 80° C., for about 30 min to about 3 hours, than at about 20° C. to about 30° C., for about 16 to about 20 additional hours.

In another embodiment, the present invention encompasses another process for preparing Form A of ramelteon comprising sonicating a solution of ramelteon in an organic solvent while cooling the solution to about 0° C. to about 11° C. to obtain Form A of ramelteon.

The term “sonication” broadly refers to a non-invasive method to improve the crystallization process. Ultrasound can impact nucleation, growth and size of the crystals. It can be used to induce nucleation in the metastable zone without seeding, avoiding primary nucleation. This technique can be used in the metastable zone or on the slurry phase to increase and modify the crystal size. The ultrasound can be applied in a continuous or in a discontinuous manner, performing several cycles.

Commercial units typically operate at around 20 kHz with multiple transducers. Average power densities for the multiple transducers are about 15 to about 80 W/L.

Suitable organic solvents include, but are not limited to, C₁-C₄ alcohols such as methanol, ethanol, and isopropanol; C₄-C₇ ethers such as tetrahydrofuran, methyl tert butyl ether, and cyclopentyl methyl ether; C₃-C₇ esters such as ethyl acetate, methyl acetate, diethyl carbonate, and dimethyl carbonate; and C₆-C₁₄ aromatic hydrocarbons such as toluene. Most preferably, the organic solvent is ethyl acetate or toluene.

Preferably, the solution is sonicated for about 5 minutes at amplitude of about 70% and pulsed at about 45 sec on and 5 sec off.

In another embodiment, the present invention encompasses an alternate process for preparing Form A of ramelteon comprising sonicating a slurry of ramelteon in the presence of an organic solvent such as methyl tert-butyl ether or n-heptane to obtain Form A of ramelteon.

Preferably, the slurry is sonicated for about 50 minutes at amplitude of about 70% and a pulse of about 45 sec on and 5 sec off.

The present invention further comprises a pharmaceutical composition comprising any of the ramelteon forms of the present invention, as described herein, and at least one pharmaceutically acceptable excipient.

The present invention comprises a pharmaceutical composition comprising any of the ramelteon forms of the present invention made by a process of the present invention as described herein, and at least one pharmaceutically acceptable excipient.

The present invention further encompasses the use any of the ramelteon forms of the present invention as described herein, for the manufacture of a pharmaceutical composition.

Methods of administration of a pharmaceutical composition of the present invention may comprise administration in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like. When the pharmaceutical composition comprises any one of the crystalline ramelteon forms of the present invention, a liquid pharmaceutical composition is a suspension or emulsion, wherein ramelteon retains its crystalline form.

Pharmaceutical compositions of the present invention can optionally be mixed with other forms of ramelteon and/or other active ingredients. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions include for example acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, or starch.

Disintegrants can increase dissolution. Disintegrants include, for example, alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Disintegration inhibitors may include, but are not limited to, white sugar, stearin, coconut butter, hydrogenated oils, and the like.

Absorption accelerators may include, but are not limited to, quaternary ammonium base, sodium lauryl sulfate, and the like.

Wetting agents may include, but are not limited to, glycerin, starch, and the like. Adsorbing agents may include, but are not limited to, starch, lactose, kaolin, bentonite, colloidal silicic acid, and the like.

A lubricant can be added to the composition to reduce adhesion and ease release of the product from a punch or dye during tableting. Lubricants include for example magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants include for example colloidal silicon dioxide, magnesium trisilicate, powdered cellulose, starch, talc and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the ramelteon of the present invention is suspended, retaining its crystalline form, together with and any other solid ingredients, which may be dissolved or suspended, in a liquid carrier, such as water, vegetable oil, alcohol, polyethylene glycol, propylene glycol or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention can also contain viscosity enhancing agents to improve the mouth-feel of the product and/or coat the lining of the gastrointestinal tract. Such agents include for example acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar can be added to improve the taste.

Preservatives and chelating agents such as alcohol, sodium benzoate, butylated hydroxy toluene, butylated hydroxyanisole and ethylenediamine tetraacetic acid can be added at safe levels to improve storage stability.

A liquid pharmaceutical composition according to the present invention can also contain a buffer such as gluconic acid, lactic acid, citric acid or acetic acid, sodium gluconate, sodium lactate, sodium citrate or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminaria, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The amount of ramelteon of the present invention contained in a pharmaceutical composition according to the present invention is not specifically restricted; however, the dose should be sufficient to treat, ameliorate, or reduce the condition.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Instruments

Powder X-ray diffraction (“XRD”) analysis can be carried out using any XRD powder diffractometer commonly used in the industry. The ramelteon samples of this invention were run in a SCINTAG powder X-ray diffractometer model X'TRA equipped with a solid-state detector. Copper radiation of λ=1.5418 Å. The sample can be introduced using a round standard aluminum sample holder with round zero background quartz plate in the bottom and is scanned by a continuous scan at a rate of 3° per minute.

### Starting Material

Ramelteon used as a starting material in the examples below was prepared by methods known in the art, for example according to the procedure described in PCT/US08/65590.

### Sonication Technique

Commercial units typically operating at around 20 kHz with multiple transducers were employed. Average power densities for the multiple transducers are about 15 to about 80 W/L.

### Spray Dry Technique

A typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream of the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. Spray-drying may be performed in a conventional manner in the processes of the present invention (see, e.g., Remington: The Science and Practice of Pharmacy, 19th ed., vol. II, pg. 1627, herein incorporated by reference). The drying gas used in the invention may be any suitable gas, although inert gases such as nitrogen, nitrogen-enriched air, and argon are preferred.

### Wet Granulation Technique

We granulation involved wetting the solid with a minimal amount of solvent, insufficient to dissolve the material, and stirring the mixture at about room temperature. Stirring can be done by tumbling or by stirring with a mechanical stirrer. Usually 0.1-0.2 ml of solvent was used per 1 gram of compound.

### Wet Grinding Technique

The method employed consisted of grinding with a pestle and mortar for at least one hour.

### Lyophilization Technique

The process comprised cooling the solution to about 25° C., and then to about −30° C., providing a frozen mass. The frozen mass is then subjected to a pressure of less then one atmosphere, sufficient to remove the solvent.

### Preparation of a Mixture of Amorphous and Form A of Ramelteon

### Example 1

Ramelteon (30 mg) was slurried in n-decane (0.9 ml) at room temperature under magnetic stirring for 18 hours. The product was isolated by decantation. The wet material was analyzed by XRD and found to be mostly amorphous ramelteon mixed with ramelteon Form A.

### Preparation of Ramelteon Form A

### Example 2

### Sonication

Crystallization: Ramelteon (1 g) was charged into the vial (20 ml) followed by addition of the appropriate solvent [EtOAc or toluene] (10 V). The vial was equipped with a sonication finger and was sonicated for approximately 5 min at amplitude: 70%; pulse: 45 sec on, 5 sec off. The solution became clear, the sonication was stopped and then the vial was cooled in the ice-water bath for 2 min. Then the sonication was recommenced at amplitude: 40%; pulse: 10 sec on, 5 sec off and the massive precipitation occurred. The solid was collected by filtration and the obtained wet material was analyzed by XRD and found to be ramelteon Form A.

Slurry: Ramelteon (1 g) was slurried in the appropriate solvent [MTBE or n-heptane] (10 V) in the vial (20 ml) equipped with a sonication finger. The slurry was sonicated for 50 min at amplitude: 70%; pulse: 45 sec on, 5 sec off. Then the sonication was stopped and the solid was collected by filtration. The obtained wet material was analyzed by XRD and found to be ramelteon Form A.

### Example 3

### Spray Dryer

Ramelteon (10 g) was dissolved in EtOH and injected into the spray dryer. The obtained dry material was analyzed by XRD and found to be ramelteon Form A.

### Example 4

### Lyophilization

Ramelteon (5 g) was dissolved in tert-BuOH (40 V) to give clear solution which was lyophilized. The obtained dry material was analyzed by XRD and found to be ramelteon Form A.

### Example 5

### Wet Grinding

Ramelteon (200 mg) was ground with mortar and pestle in the presence of a few drops of the following solvent: H₂O or EtOH or IPA or n-heptane. The obtained wet material was analyzed by XRD and found to be ramelteon Form A.

### Example 6

### Wet Granulation

Ramelteon (100 mg) was wet granulated in the presence of 2 drops of H₂O, EtOH, IPA or n-heptane, by tumbling the wet solids in a flask mounted on a rotary evaporator. The obtained wet material was analyzed by XRD and found to be ramelteon Form A.

### Example 7

### Melting

(a) Ramelteon (100 mg) was melted by heating in a flask at 123° C. The flask was allowed to cool to room temperature (RT) and the obtained dry material was analyzed by XRD and found to be ramelteon Form A.

(b) Ramelteon (100 mg) was melted by heating in a flask at 123° C. followed by addition of ice. The obtained solid was collected by filtration and the wet material was analyzed by XRD and found to be ramelteon Form A.

### Example 8

### Crystallization—Experimental Procedure I

Ramelteon (30 mg) was slurried in Solvent I at room temperature for a period of time to give a clear solution (Table 1). Then H₂O was added and precipitation occurred. The product was isolated by vacuum filtration and the wet material was analyzed by XRD and found to be ramelteon Form A.

TABLE 1

| Solvent I | H₂O | time |
|---|---|---|
| PGME (3 V) | 3 V | 65 h |
| propylene | 10 V | 65 h |
| glycol (10 V) | | |
| MeCN (3 V) | 3 V | 18 h |
| DMF (10 V) | 10 V | 65 h |

### Example 9

### Crystallization—Experimental Procedure II

Step A: Ramelteon (200 mg) was dissolved in Solvent I at temperature A followed (optionally) by addition of Solvent II and (optionally) Solvent II. (Table 2).

Step B: The reaction mixture was stirred at temperature B for period of time B and the obtained precipitate was collected by filtration. The obtained wet material was analyzed by XRD and found to be ramelteon Form A.

Step C: The wet material was dried in the oven at temperature C for period of time C to give dry material which was analyzed by XRD and found to be ramelteon Form A.

| Step A: dissolving step | | | | Step B: cooling | | | Step C: drying | |
|---|---|---|---|---|---|---|---|---|
| Solvent I | Solvent II | Solvent III | Temp. A | Temp. B | Time B | | Temp. C | Time C |
| EtOAc (20 V) | — | — | RT | 0° C. | 30 | min | 40° C. | 24 h |
| EtOAc (12.5 V) | — | — | 72° C. | RT | 1 | h | 50° C. | 20 h |
| EtOH (15 V) | H₂O (15 V) | — | 80° C. | RT | 1 | h | 50° C. | 22 h |
| MeOH (10 V) | H₂O (7.5 V) | — | RT | RT | 1 | min | 50° C. | 21 h |
| Toluene (5 V) | n-heptane (5 V) | — | RT | RT | 30 | min | 50° C. | 17 h |
| Acetone (15 V) | H₂O (25 V) | — | RT | RT | 10 | min | 50° C. | 24 h |
| Acetone (15 V) | n-heptane (45 V) | — | RT | 0° C. | 1 | h | 50° C. | 22 h |
| IPA (15 V) | n-heptane (60 V) | — | RT | 5° C. | 17 | h | 50° C. | 21 h |
| IPA (2.5 V) | Et₂O (25 V) | — | RT | 0° C. | 1 | h | — | — |
| EtOAc (21 V) | Et₂O (20 V) | — | RT | 0° C. | 40 | min | 50° C. | 24 h |
| MIBK (20 V) | n-heptane (40 V) | — | RT | RT | 30 | min | 35° C. | 24 h |
| Acetone (10 V) | iPr₂O (20 V) | — | RT | 0° C. | 30 | min | 35° C. | 24 h |
| IPA (10 V) | H₂O (15 V) | — | RT | RT | 40 | min | 50° C. | 21 h |
| MIBK (15 V) | Et₂O (10 V) | — | RT | 0° C. | 30 | min | 50° C. | 21 h |
| DMSO (3.75 V) | H₂O (6 drops) | — | RT | RT | 30 | min | 50° C. | 16 h |
| AcOH (3.75 V) | H₂O (5 V) | — | RT | RT | 30 | min | 50° C. | 16 h |
| DCM (10 V) | MeCycHx (35 V) | — | RT | 0° C. | 30 | min | 50° C. | 22 h |
| CHCl3 (7.5 V) | MeCycHx (35 V) | — | RT | 0° C. | 30 | min | 50° C. | 22 h |
| THF (7.5 V) | H₂O (20 V) | — | 75° C. | RT | 16 | h | 50° C. | 25 h |
| THF (10 V) | MTBE (35 V) | — | RT | 5° C. | 17 | h | 50° C. | 24 h |
| EtOH (1.25 V) | EtOAc (10 V) | MeCycHx (25 V) | 76° C. | 0° C. | 30 | min | 50° C. | 22 h |
| Toluene (10 V) | DMSO (0.5 V) | MeCycHx (25 V) | 81° C. | RT | 15 | min | 50° C. | 22 h |
| Et lactate (5 V) | MeCycHx (50 V) | — | RT | RT | 15 | min | 50° C. | 16 h |
| MeCN (10 V) | — | — | 80° C. | RT | 15 | min | — | — |
| CO(OMe)₂ (10 V) | — | — | 80° C. | RT | 15 | min | 50° C. | 16 h |
| CO(OEt)₂ (10 V) | — | — | 80° C. | RT | 15 | min | 50° C. | 16 h |

### Example 10

### Evaporation—Experimental Procedure I

Step A: Ramelteon (200 mg) was dissolved in Solvent I at temperature A followed (optionally) by addition of Solvent II, and the obtained clear solution was stirred for period of time A (Table 3).

Step B: The solvent was removed by evaporation and the obtained wet material was analyzed by XRD and found to be ramelteon Form A.

Step C: The wet material was dried in the oven at temperature C for period of time C to give dry material which was analyzed by XRD and found to be ramelteon Form A.

TABLE 3

| Step A | | | | | Step C | |
|---|---|---|---|---|---|---|
| | | Temp. | Time | | Temp. | Time |
| Solvent I | Solvent II | A | A | | C | C |
| MeOAc (70 V) | — | RT | 5 | min | 40° C. | 16 h |
| EtOAc (20 V) | — | RT | 5 | min | 40° C. | 24 h |
| MeOH (10 V) | iPr₂O (165 V) | RT | 24 | h | 35° C. | 24 h |
| IPA (15 V) | n-heptane (60 V) | RT | 24 | h | 50° C. | 21 h |
| EtOAc (21 V) | Et₂O (20 V) | RT | 15 | min | 50° C. | 24 h |
| Acetone (10 V) | iPr₂O (20 V) | RT | 15 | min | 35° C. | 24 h |
| MIBK (20 V) | H₂O (10 V) | RT | 48 | h | 50° C. | 21 h |
| IPA (10 V) | H₂O (15 V) | RT | 15 | min | 50° C. | 21 h |
| CH₂Cl₂ (7.5 V) | MTBE (125 V) | RT | 48 | h | 50° C. | 25 h |
| CHCl₃ (7.5 V) | MTBE (115 V) | RT | 48 | h | 50° C. | 25 h |
| Acetone (20 V) | — | RT | 15 | min | — | — |
| CH₂Cl₂ (30 V) | — | RT | 15 | min | — | — |
| CHCl₃ (30 V) | — | RT | 15 | min | — | — |
| Et₂O (160 V) | — | RT | 15 | min | — | — |
| THF (10 V) | MTBE (35 V) | RT | 15 | min | 50° C. | 25 h |

### Example 11

### Evaporation—Experimental Procedure II

Ramelteon (30 mg) was slurried in Solvent at the indicated Temperature for the indicated period of time to give a clear solution (Table 4). The solvent was removed by evaporation and the obtained wet material was analyzed by XRD and found to be ramelteon Form A.

TABLE 4

| | Volume Ratio | Temperature | |
|---|---|---|---|
| Solvent | (ml/g) | [° C.] | Time |
| Methyl-ethyl ketone | 7 V | RT | 65 h |
| Dioxane | 3 V | RT | 65 h |
| EtOH | 10 V | RT | 65 h |
| 1-Propanol | 10 V | RT | 65 h |
| Cellosolve ™ | 7 V | RT | 65 h |
| MIBK | 10 V | RT | 65 h |
| Isopropyl-methyl | 3 V | RT | 18 h |
| ketone | | | |
| N-Methyl-pyrrolidone | 3 V | RT | 18 h |

| | | |
|---|---|---|
| Dimethyl carbonate | 3 V | 80° C. (1.5 h) + RT (18 h) |

### Preparation of a Substantially Amorphous Ramelteon Form

### Example 12

Ramelteon (30 mg) was slurried in water/propylene glycol monomethyl ether 1:1 (1.8 ml) at 80° C., under magnetic stirring for 1.5 hours, and another 18 hours at room temperature. The solid was collected by decantation and the wet material was analyzed by XRD and found to be substantially amorphous ramelteon, having the PXRD pattern shown in FIG. 2.

1. A method of preparing a crystalline form of ramelteon characterized by powder XRD pattern peaks at about 7.6, 8.0, 14.2, 14.7, and 16.8±0.2 degrees two theta, comprising providing a solution of ramelteon in an organic solvent selected from the group consisting of propylene glycol monoethyl ether, propylene glycol, acetonitrile, dimethyl formamide, acetic acid, isopropanol, acetone, methanol, dimethyl sulfoxide, and tetrahydrofuran; and precipitating the ramelteon by adding water.

2-12. (canceled)

13. A method of preparing a crystalline form of ramelteon characterized by powder XRD pattern peaks at about 7.6, 8.0, 14.2, 14.7, and 16.8±0.2 degrees two theta, comprising crystallizing ramelteon from an organic solvent selected from the group consisting of acetonitrile, dimethyl carbonate, and diethyl carbonate.

14-17. (canceled)

18. A method of preparing a crystalline form of ramelteon characterized by powder XRD pattern peaks at about 7.6, 8.0, 14.2, 14.7, and 16.8±0.2 degrees two theta, comprising providing a solution of ramelteon in an organic solvent selected from the group consisting of toluene, acetone, isopropanol, ethyl acetate, methyl isobutyl ketone, dimethyl sulfoxide, dichloromethane, chloroform, ethanol, and tetrahydrofuran; and combining the solution with an anti-solvent selected from the group consisting of n-heptane, diethyl ether, diisopropyl ether, methyl cyclohexane, and methyl tert-butyl ether, to precipitate the crystalline form.

19. The method of claim 18, wherein the mixture of solvent and anti-solvent is selected from the group consisting of: toluene/n-heptane, acetone/n-heptane, isopropanol/n-heptane, isopropanol/diethyl ether, ethyl acetate/diethyl ether, methyl isobutyl ketone/n-heptane, acetone/diisopropyl ether, methyl isobutyl ketone/diethyl ether, dichloromethane/methyl cyclohexane, chloroform/methyl cyclohexane, tetrahydrofuran/methyl tert-butyl ether, ethyl acetate/methyl cyclohexane, and dimethyl sulfoxide/methyl cyclohexane.

20-27. (canceled)

28. The method of claim 18, wherein the solvent is tetrahydrofuran.

29. (canceled)

30. A method of preparing a crystalline form of ramelteon characterized by powder XRD pattern peaks at about 7.6, 8.0, 14.2, 14.7, and 16.8±0.2 degrees two theta, comprising providing a solution of ramelteon in an organic solvent, or a mixture of organic solvents, and removing the solvents.

31. The method of claim 30, wherein the solvent is removed by spray drying.

32. The method of claim 31, wherein the solvent is selected from the group consisting of: C₁-C₈ alcohols, C₃-C₈ ketones, C₄-C₈ ethers, C₃-C₈ esters, C₁-C₃ aliphatic nitrites, and C₂-C₆ aliphatic amines.

33. The method of claim 32, wherein the solvent is a C₁-C₈ alcohol.

34. The method of claim 33, wherein the C₁-C₉ alcohol is selected from the group consisting of ethanol, isopropanol, and methanol.

35-46. (canceled)

47. A method of preparing a crystalline form of ramelteon characterized by powder XRD pattern peaks at about 7.6, 8.0, 14.2, 14.7, and 16.8±0.2 degrees two theta, the process comprising wet granulating ramelteon in the presence of a solvent selected from the group consisting of water, ethanol, isopropanol, and n-heptane.

48-51. (canceled)

52. A method of preparing a crystalline form of ramelteon characterized by powder XRD pattern peaks at about 7.6, 8.0, 14.2, 14.7, and 16.8±0.2 degrees two theta, the process comprising sonicating a solution of ramelteon in an organic solvent selected from the group consisting of ethyl acetate and toluene to form a precipitate of crystalline ramelteon.

53. The method of claim 52, wherein the solution is cooled to about 0° C. before collecting the precipitate.

54-75. (canceled)
